(19) United States
(12) Patent Application Publication
(10) Pub. No.: US 2026/0074068 A1
(43) Pub. Date: Mar. 12, 2026

(54) SYSTEMS AND METHODS FOR MOBILE APPLICATION CONTROL OF INFUSION PUMPS

(71) Applicant: Tandem Diabetes Care, INC, San Diego, CA (US)

(72) Inventors: Sydney Choi, San Diego, CA (US); Timothy Sloncz, San Diego, CA (US); Yati Bawri, San Diego, CA (US); Hansa Nayaran, San Diego, CA (US)

(21) Appl. No.: 19/252,256

(22) Filed: Jun. 27, 2025

Related U.S. Application Data
(60) Provisional application No. 63/665,506, filed on Jun. 28, 2024.

(51) Int. Cl. G16H 40/67; A61M 5/142

(52) U.S. Cl. CPC G16H 40/67; A61M 5/142; A61M 2205/502

(57) ABSTRACT

Having two active software applications for control of or otherwise communicating with different infusion pumps on the same control device presents a safety concern. For example, a user may open up the wrong software application and program a bolus of medicament for delivery with one pump thinking that the user was programming the bolus for delivery by the other pump. A user could also review data in the wrong software application and make other therapy decisions based on data that is for a different pump or that is outdated because the application is currently connected with the pump. Disclosed herein are systems and methods for preventing such errors.

### PRIORITY CLAIM

The present application claims the benefit of U.S. Provisional Application No. 63/665,506 filed Jun. 28, 2024, which is hereby incorporated herein by reference in its entirety.

### TECHNICAL FIELD

The present disclosure relates generally to ambulatory infusion pump systems and, more particularly, to programming of ambulatory infusion pumps.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art. One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

With the proliferation of handheld electronic devices, such as mobile phones (e.g., smartphones), there is a desire to be able to remotely utilize such devices, as well as dedicated wireless controllers designed to work with one or more infusion pumps and/or types of infusion pumps, to optimize usage of infusion pumps. These remote controllers would enable a pump to be monitored, programmed and/or operated more privately, more conveniently and more comfortably. Accordingly, one potential use of dedicated remote devices and handheld consumer electronic devices (such as smartphones, tablets and the like) is to utilize such devices as controllers for remotely programming, operating and/or otherwise communicating with infusion pumps. However, in situations where an infusion pump can be programmed from more than one device, including the pump itself, there can be issues with a pump receiving commands or other communications from multiple devices at or about the same time.

In addition, different types of infusion pumps exist, including different types of pumps provided by the same company. Each type of pump may have a different user interface, mobile application, or other means of controlling the pump. There are also other devices such as continuous glucose monitors that may have mobile applications that can interface with, communicate with, and/or control pumps. For a user that owns multiple pumps and/or related devices, it is important that the user not attempt to program a pump with software intended for control or access to another device or to otherwise rely on such software in making therapy decisions if the software doesn't have the current pump and/or user information.

### SUMMARY

Having two active software applications for control of different infusion pumps on the same control device presents a safety concern. For example, a user may open up the wrong software application and program a bolus of medicament for delivery with one pump thinking that the user was programming the bolus for delivery by the other pump. A user could also review data in the wrong software application and make other therapy decisions based on data that is for a different pump. Disclosed herein are systems and methods for preventing such errors. In particular, systems and methods for mobile application control of infusion pumps disclosed herein can include disabling use of a first pump software application when a second pump software application is downloaded onto a given device.

The systems and methods disclosed herein also have broader applicability to other medical device software applications. For example, there may be a software application relating to the user's infusion pump and one or more other software applications related to a continuous glucose monitor or other diabetes management device as well as one or more software applications relating to diabetes data management and monitoring downloadable onto a user's phone. In the context of a software application designed to interface with a continuous glucose monitor (CGM), for example, the infusion pump software application may already be interfacing with and receiving data from the CGM device. In this circumstance, the CGM application may not be connected with the CGM device. If the user accesses the CGM's software application instead of the pump software application, the user may receive outdated information that could cause the user to deliver inappropriate therapy with the pump.

In an embodiment, a method of managing mobile application control of one or more infusion pumps can include determining that a first pump software application for remote control of an infusion pump is operating on a user device. If it is detected that a second pump software application for remote control of an infusion pump has been downloaded onto the user device operation of the first pump software application on the user device can be disabled.

In an embodiment, a method of managing mobile applications relating to a user's management of the user's diabetes can include determining that a first software application relating to diabetes management is operating on a user device. If it is detected that a second software application relating to diabetes management has been downloaded onto the user device, operation of the first software application relating to diabetes management on the user device can be disabled.

### BRIEF DESCRIPTION OF THE DRAWINGS

The disclosure may be more completely understood in consideration of the following detailed description of various embodiments of the disclosure in connection with the accompanying drawings, in which:

FIG. 1 is a medical device that can be used with embodiments of the disclosure.

FIG. 2 is a block diagram representing a medical device that can be used with embodiments of the disclosure.

FIGS. 3A-3B depict an alternate embodiment of a pump system according to the disclosure.

FIGS. 4A-4B depict remote control devices for an infusion pump system according to the disclosure.

FIGS. 5A-5B depict a remote control device according to the disclosure.

FIG. 6 depicts a display screen for a remote control device according to the disclosure.

FIG. 7 depicts a push notification for a remote control device according to the disclosure.

FIG. 8 depicts a flowchart of steps in a method of managing mobile application usage in an infusion pump system according to the disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the disclosure.

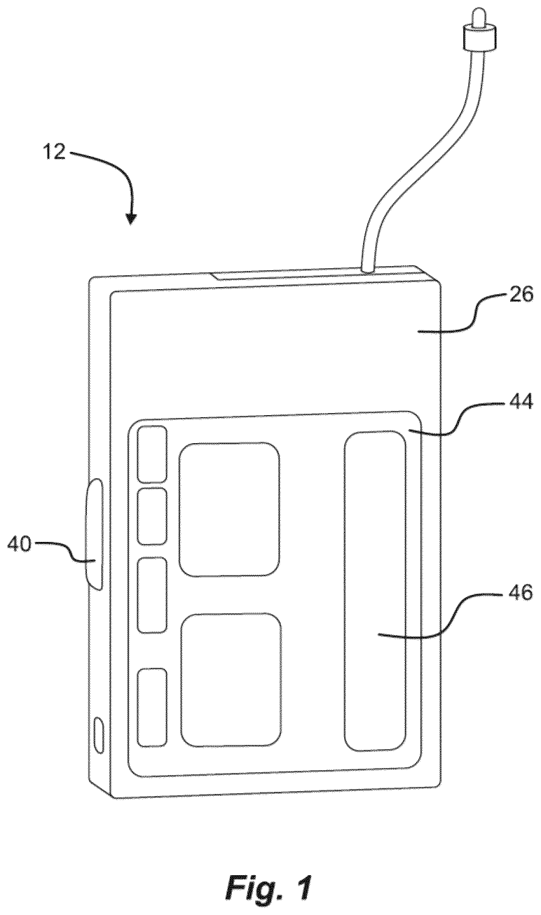

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicaments to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

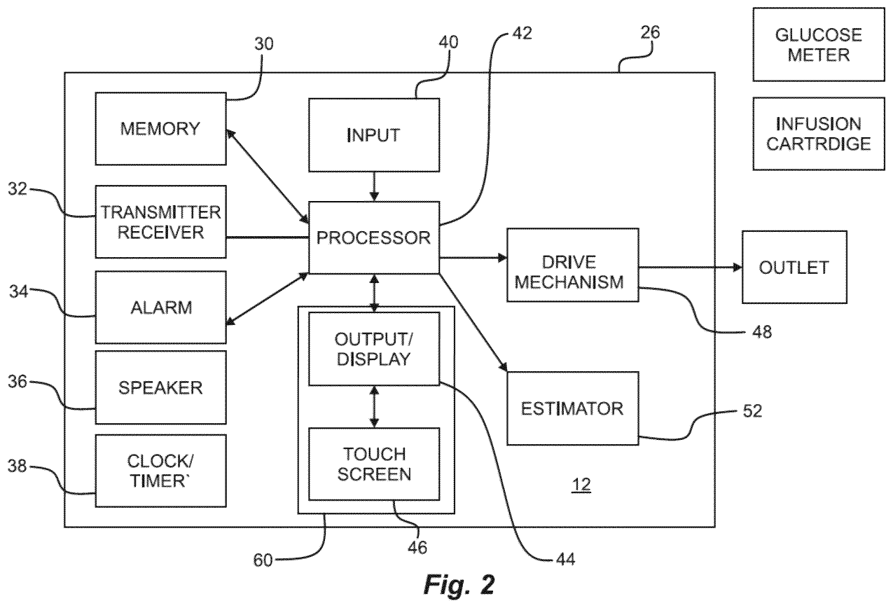

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also be programmed to receive signals and/or other data from an input device, such as, by way of example, a pressure sensor, a temperature sensor, accelerometer, GPS receiver, or the like.

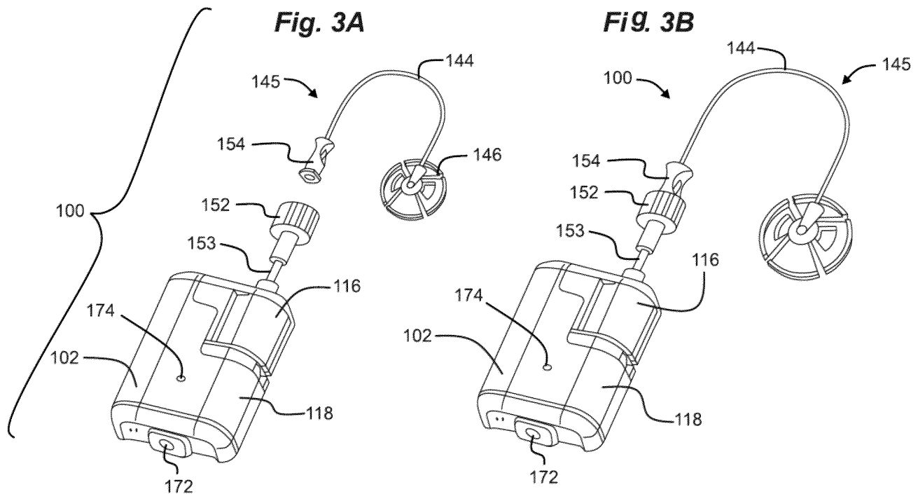

FIGS. 3A-3B depict another pump system including a pump 102 that can be used with embodiments. Drive unit 118 of pump 102 includes a drive mechanism 122 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment pump 102 does not include a display but may include one or more indicator lights 174 and/or one or more input buttons 172. Pump 102 can also incorporate any or all of the features described with respect to pump 12 in FIG. 2. Further details regarding such pumps can be found in U.S. Pat. No. 10,279,106 and U.S. Patent Publication Nos. 2016/0339172 and 2017/0049957, each of which is hereby incorporated herein by reference in its entirety.

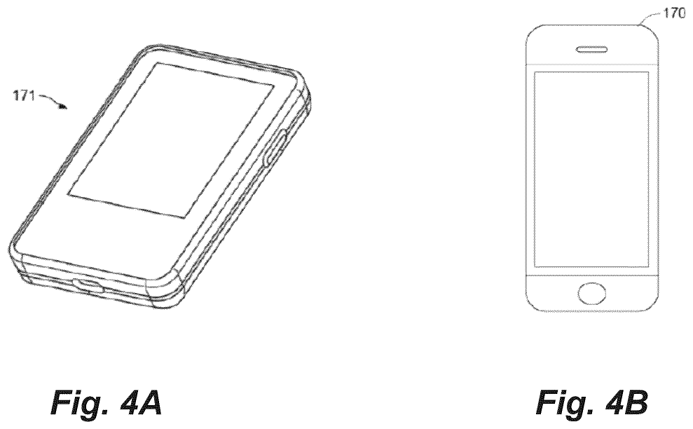

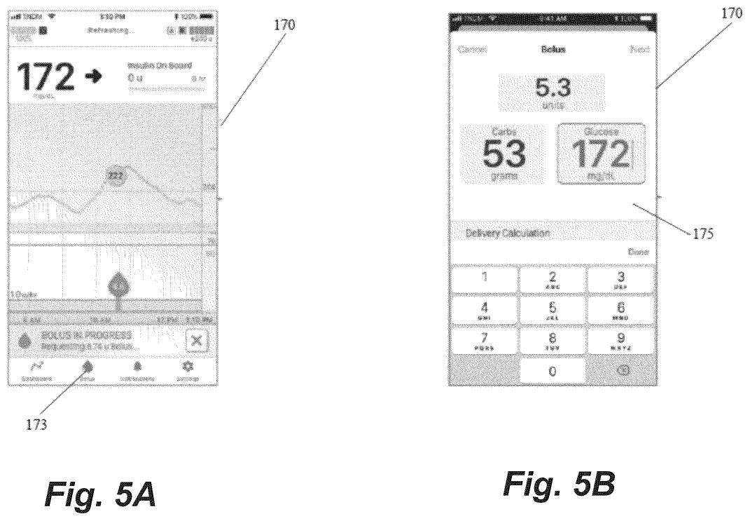

Referring to FIGS. 4A-4B, one or more remote control devices 170, 171 can be used to communicate with the processor of pump 12 or pump 102 to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smartphone executing a software application for control of the pump, a dedicated remote controller 171, a wearable electronic watch or electronic health or fitness monitor or personal digital assistant (PDA), etc., or a tablet, laptop or personal computer. For example, a bolus programming interface such as screen 175 depicted in FIG. 5B may be able to be displayed on a smartphone or other remote control device by selecting a bolus icon 173 on a home screen of a software application such as depicted in FIG. 5A. Any other programming tasks that can otherwise be carried out with a user interface of a pump (for a pump with a user interface) can instead be accomplished using the software application executing on the remote control device 170.

In certain circumstances, an individual user or a family may have more than one infusion pump, such as a larger pump having a touchscreen display like pump 12 that can be controlled from the pump or with a software application operating on a remote control device and a smaller, patch-style pump without a display such as pump 102 intended to be primarily remote controlled. For example, an adult in the family may have one type of pump 12 and a child may have another type of pump 102. Alternatively, an individual user may have multiple pumps that are utilized for different activities (e.g., one type of pump when going to work and another when exercising). Typically, these different styles of pumps will have separate software applications that are downloaded onto the user's phone or other remote control device for control of each pump. There may also be more than one software application available to download for control of a single type of pump.

Having two active software applications for control of different pumps on the same control device presents a safety concern. For example, a user may open up the wrong software application and program a bolus for delivery with one pump thinking that the user was programming the bolus for delivery by the other pump. A user could also review data in the wrong software application and make other therapy decisions based on data that is for a different pump. A similar concern may be presented if there is more than one different software application for control of the same pump on a user device, particularly if one of the software applications is not currently paired with the pump or otherwise doesn't have the most recent data for the pump. Disclosed herein are systems and methods addressing such issues.

In particular, the systems and methods disclosed herein only enable a given phone or other remote control device to have only one software application for control of a pump installed and active on the user's device. For example, if the user installs a second application for a control of a pump onto a device that already has a first pump application, the first pump application is automatically disabled. This may include automatically logging the user out of the first pump application and/or automatically unpairing the first pump application from the pump.

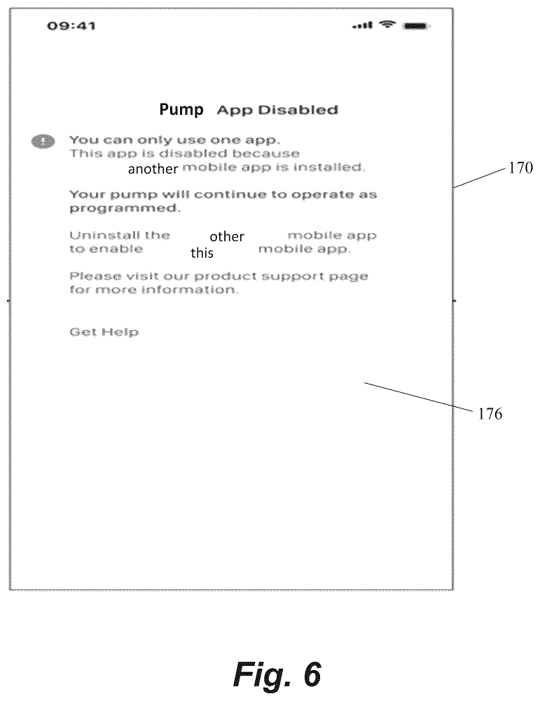

FIG. 6 depicts an App Disabled screen 176 that can be presented on the display of a remote control device 170 such as a phone in such a circumstance. App Disabled screen 176 can inform the user that only one app is allowed on the device and that the first pump application has been disabled now that the second pump application has been downloaded. The pump corresponding to the first application may continue to operate as programmed previously programmed for ongoing therapy (i.e., basal delivery), but the first pump application can no longer interact with the pump. In other words, disabling of a software application for control of an infusion pump does not have any effect on the functionality of the pump itself. The user may further be notified that in order for the first pump application to be enabled, the second pump application must be uninstalled. In some embodiments, App disabled screen 176 can be the only screen that can be displayed in the first pump application on the device such that the user is not able to otherwise navigate through or operate any aspect of the first pump application. In other embodiments, the first pump application may further be automatically uninstalled from the device after the second pump application is installed.

Disabling of the first pump application can result in one or more of: 1) not being able to view pump data in the application; 2) not being able to program a bolus from the application; 3) no alerts, alarms, reminders, or malfunctions relating to the pump will be viewable in the application; and 4) wireless data uploads to or from the application will be disabled.

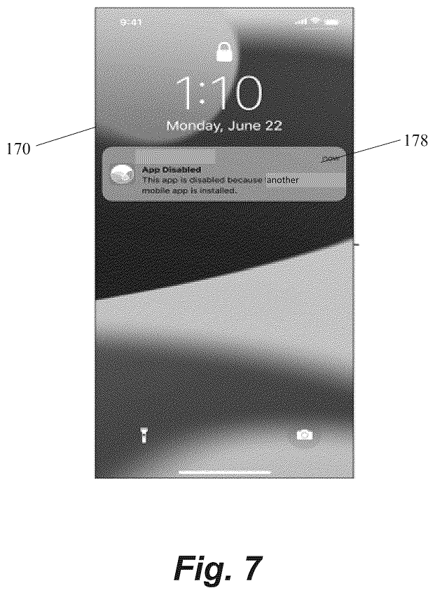

In some embodiments, in addition to providing a notification to the user within the first pump application that the application has been disabled, a push notification can be provided on the user's device. One example of such a push notification 178 provided on a remote control device 170 such as a phone is depicted in FIG. 7. Notification 178 can inform the user that the first pump application has been disabled due to the second pump application having been installed. If the user selects the notification 178, the first pump application can be opened and a screen such as App Disabled screen 176 providing further details may be displayed on the device.

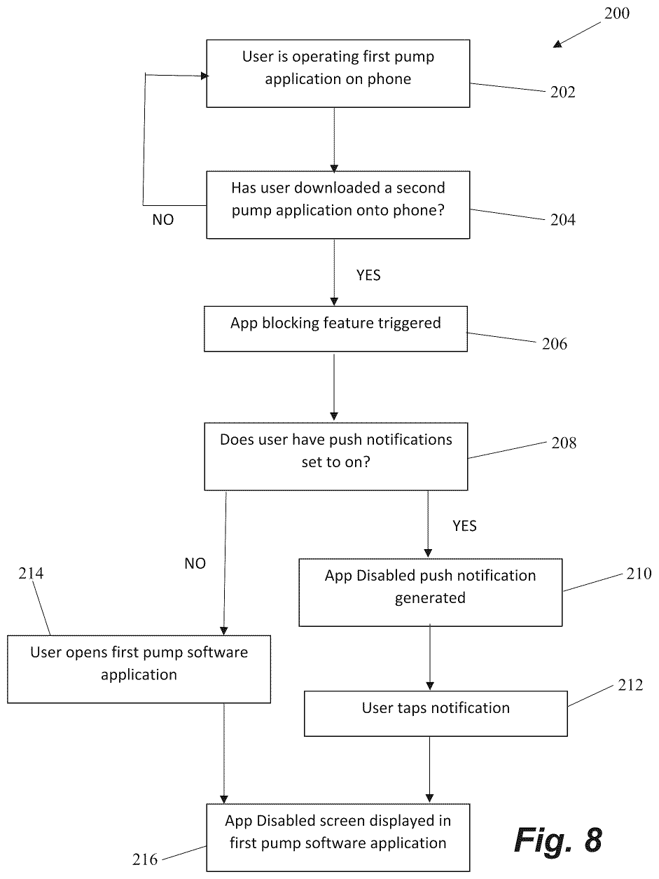

FIG. 8 depicts a flowchart of steps in a method 200 of managing use of infusion pump software applications according to the disclosure. At step 202, a user has an existing, first pump software application downloaded onto the user's phone for operation of an infusion pump. At step 204, it is determined whether the user has downloaded a second pump software application onto the phone. If the user has not downloaded a second pump software application, no changes are made and a user will continue to operate the first pump software application on the user's phone. If the user has downloaded a second pump software application, an app blocking feature is triggered at step 206 that will restrict usage of the first pump software application. In embodiments, the app blocking feature can sign the user out of the first pump software application and unpair the application from the corresponding pump. At step 208, it is determined if the user has push notifications set to on. If push notifications are set to on, at step 210 the App Disabled push notification 176 of FIG. 7 is generated and displayed on the user's phone. If the user subsequently taps the push notification 178 at step 212, the first pump software application will open and display the App Disabled screen 176 of FIG. 6 at step 216. If push notifications are not set to on, the App Disabled screen 176 of FIG. 6 will be displayed to the user at step 216 after the user next selects the first pump software application on the user's phone at step 214. In order to re-enable the first pump software application, the user will need to delete the second pump software application from the user's phone. At that point, the user can log back into the first pump software application and re-pair the user's pump with the application.

Although primarily described herein with respect to software applications for control of different infusion pumps, the systems and methods disclosed herein also have broader applicability to other medical device software applications. For example, there may be a software application relating to the user's infusion pump and one or more other software applications related to a continuous glucose monitor or other diabetes management device as well as one or more software applications relating to diabetes data management and monitoring downloadable onto a user's phone. In the context of a software application designed to interface with a continuous glucose monitor (CGM), for example, the infusion pump software application may already be interfacing with and receiving data from the CGM device. In this circumstance, the CGM application may not be connected with the CGM device. If the user accesses the CGM's software application, the user may receive outdated information that could cause the user to deliver inappropriate therapy with the pump.

In embodiments, a method of managing mobile application control of one or more infusion pumps can include determining that a first pump software application for remote control of an infusion pump is operating on a user device. If it is detected that a second pump software application for remote control of an infusion pump has been downloaded onto the user device operation of the first pump software application on the user device can be disabled.

In some embodiments, disabling operation of the first pump software application on the user device includes automatically logging out of the first pump software application.

In some embodiments, disabling operation of the first pump software application on the user device includes automatically unpairing the first pump software application from the infusion pump.

In some embodiments, disabling operation of the first pump software application on the user device includes automatically uninstalling the first pump software application from the user device.

In some embodiments, a notification can be provided on a user interface of the user device that the first pump software application has been disabled.

In some embodiments, the notification indicates that for the first pump software application to be reenabled the second pump software application must be uninstalled from the user device.

In some embodiments, the notification is a screen within the first pump software application.

In some embodiments, the notification is the only screen that can be displayed within the first pump software application such that the user cannot navigate among menu screens in the first pump software application.

In some embodiments, the notification is provided as a push notification on the user device.

In some embodiments, the first pump software application and the second pump software application are configured for control of different types of infusion pumps.

In embodiments, a method of managing mobile applications relating to a user's management of the user's diabetes can include determining that a first software application relating to diabetes management is operating on a user device. If it is detected that a second software application relating to diabetes management has been downloaded onto the user device, operation of the first software application relating to diabetes management on the user device can be disabled.

In some embodiments, at least one of the first software application relating to diabetes management and the second software application relating to diabetes management is a pump software application for remote control of an infusion pump.

In some embodiments, both of the first software application relating to diabetes management and the second software application relating to diabetes management are pump software applications for remote control of an infusion pump.

In some embodiments, the first pump software application and the second pump software application are configured for control of different types of infusion pumps.

In some embodiments, one of the first software application relating to diabetes management and the second software application relating to diabetes management is a software application for a continuous glucose monitor.

In some embodiments, disabling operation of the first software application relating to diabetes management on the user device includes automatically logging out of the first software application relating to diabetes management.

In some embodiments, disabling operation of the first software application relating to diabetes management on the user device includes automatically unpairing the first software application relating to diabetes management from an infusion pump.

In some embodiments, disabling operation of the first software application relating to diabetes management on the user device includes automatically uninstalling the first software application relating to diabetes management from the user device.

In some embodiments, a notification is provided on a user interface of the user device that the first software application relating to diabetes management has been disabled.

In some embodiments, the notification indicates that for the first software application relating to diabetes management to be reenabled the second software application relating to diabetes management must be uninstalled from the user device.

Although the embodiments herein have been specifically described with respect to an ambulatory infusion pump, the inventions disclosed herein could be employed with any other type of programmable medical device capable of receiving and executing remote commands. Such devices include, for example, non-ambulatory pumps, implantable pumps, defibrillators, spinal cord stimulation systems, etc. Embodiments could further include non-medical applications.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; Ser. No. 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; and 12,214,159 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164; 19/119,554; 19/134,333; 19/205,083; 19/220,426; 19/221,933; and 19/225,150.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A method of managing mobile application control of one or more infusion pumps, comprising:
determining that a first pump software application for remote control of an infusion pump is operating on a user device;
detecting that a second pump software application for remote control of an infusion pump has been downloaded onto the user device; and
disabling operation of the first pump software application on the user device in response to detecting that the second pump software application has been downloaded onto the user device.

2. The method of claim 1, wherein disabling operation of the first pump software application on the user device includes automatically logging out of the first pump software application.

3. The method of claim 1, wherein disabling operation of the first pump software application on the user device includes automatically unpairing the first pump software application from the infusion pump.

4. The method of claim 1, wherein disabling operation of the first pump software application on the user device includes automatically uninstalling the first pump software application from the user device.

5. The method of claim 1, further comprising providing a notification on a user interface of the user device that the first pump software application has been disabled.

6. The method of claim 5, wherein the notification indicates that for the first pump software application to be reenabled the second pump software application must be uninstalled from the user device.

7. The method of claim 5, wherein the notification is a screen within the first pump software application.

8. The method of claim 7, wherein the notification is the only screen that can be displayed within the first pump software application such that the user cannot navigate among menu screens in the first pump software application.

9. The method of claim 5, wherein the notification is provided as a push notification on the user device.

10. The method of claim 1, wherein the first pump software application and the second pump software application are configured for control of different types of infusion pumps.

11. A method of managing mobile applications relating to a user's management of the user's diabetes, comprising:
determining that a first software application relating to diabetes management is operating on a user device;
detecting that a second software application relating to diabetes management has been downloaded onto the user device; and
disabling operation of the first software application relating to diabetes management on the user device in response to detecting that the second software application relating to diabetes management has been downloaded onto the user device.

12. The method of claim 11, wherein at least one of the first software application relating to diabetes management and the second software application relating to diabetes management is a pump software application for remote control of an infusion pump.

13. The method of claim 12, wherein both of the first software application relating to diabetes management and the second software application relating to diabetes management are pump software applications for remote control of an infusion pump.

14. The method of claim 13, wherein the first pump software application and the second pump software application are configured for control of different types of infusion pumps.

15. The method of claim 12, wherein one of the first software application relating to diabetes management and the second software application relating to diabetes management is a software application for a continuous glucose monitor.

16. The method of claim 11, wherein disabling operation of the first software application relating to diabetes management on the user device includes automatically logging out of the first software application relating to diabetes management.

17. The method of claim 11, wherein disabling operation of the first software application relating to diabetes management on the user device includes automatically unpairing the first software application relating to diabetes management from an infusion pump.

18. The method of claim 11, wherein disabling operation of the first software application relating to diabetes management on the user device includes automatically uninstalling the first software application relating to diabetes management from the user device.

19. The method of claim 11, further comprising providing a notification on a user interface of the user device that the first software application relating to diabetes management has been disabled.

20. The method of claim 19, wherein the notification indicates that for the software application relating to diabetes management to be reenabled the second software application relating to diabetes management must be uninstalled from the user device.
